(19) United States
(12) Patent Application Publication
(10) Pub. No.: US 2005/0268406 A1
(43) Pub. Date: Dec. 8, 2005

(54) Compositions for dyeing keratin fibers, comprising at least one alcohol oxidase and at least one oxyalkylenated anionic surfactant, and processes using these compositions

(76) Inventor: Gregory Plos, Tokyo (JP)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/044,082

(22) Filed: Jan. 28, 2005

Related U.S. Application Data
(60) Provisional application No. 60/545,570, filed on Feb. 19, 2004.

(30) Foreign Application Priority Data
Jan. 28, 2004 (FR) 04 00781

(51) Int. Cl. A61K 7/13

(52) U.S. Cl. 8/406

(57) ABSTRACT

The present disclosure relates to compositions for dyeing keratin fibers, for example human keratin fibers, such as the hair, comprising, in a medium that is suitable for dyeing, at least one oxidation dye precursor, at least one alcohol oxidase enzyme, at least one substrate for the enzyme, and at least one oxyalkylenated anionic surfactant. The present disclosure also relates to processes for dyeing keratin fibers, which comprise applying the compositions disclosed herein, and to dyeing “kits” comprising the compositions as disclosed herein.

This application claims benefit of U.S. Provisional Application No. 60/545,570, filed Feb. 19, 2004.

The present disclosure relates to compositions for dyeing keratin fibers, for example human keratin fibers, such as the hair, comprising, in a medium that is suitable for dyeing, at least one oxidation dye precursor, at least one alcohol oxidase enzyme, at least one substrate for the enzyme, and at least one oxyalkylenated anionic surfactant.

It is known to dye keratin fibers, such as human hair, with dye compositions containing oxidation dye precursors, for example ortho- or para-phenylenediamines, ortho- or para-aminophenols, and heterocyclic compounds, which are generally referred to as oxidation bases. These oxidation bases are colorless or weakly colored compounds which, when combined with oxidizing products, may give rise to colored compounds by a process of oxidative condensation.

It is also known that the shades obtained with these oxidation bases may be varied by combining them with couplers or coloration modifiers, the latter being chosen, for example, from aromatic meta-diamines, meta-aminophenols, meta-diphenols, and certain heterocyclic compounds, such as indole compounds.

The variety of molecules used as oxidation bases and couplers makes it possible to obtain a wide range of colors.

The “permanent” coloration obtained by means of these oxidation dyes should generally satisfy certain requirements. First, it should have substantially no toxicological drawbacks. Second, it should allow shades of the desired intensity to be obtained. Third, it should have good resistance to external agents such as light, bad weather, washing, permanent waving, perspiration, and rubbing.

The dyes should also allow white keratin fibers to be covered. They should also be as unselective as possible; that is to say that they should allow the smallest possible differences in coloration to be produced over the entire length of the same keratin fiber, which is generally differently sensitized (i.e. damaged) between its end and its root.

Dyeing is generally performed in strongly alkaline medium, in the presence of hydrogen peroxide. However, the use of alkaline media in the presence of hydrogen peroxide can have the drawbacks of causing considerable degradation of the fibers, and bleaching of keratin fibers, which is not always desirable.

Oxidation dyeing of keratin fibers may also be performed using oxidizing systems other than hydrogen peroxide, such as enzymatic systems. Thus, French Patent Application No. FR 2,769,219 describes the use of a uricase enzyme and of its uric acid substrate in oxidation dyeing to dye keratin fibers. European Patent Application No. EP-A-0,310,675 describes the use of oxidation dye precursors of benzenic type in combination with enzymes such as pyranose oxidase and glucose oxidase. The more recent French Patent Application FR 2,833,492 describes the use of the alcohol oxidase enzyme as the sole enzyme in an oxidation dye composition for dyeing keratin fibers.

The aim of the present disclosure is to provide novel compositions for dyeing keratin fibers by oxidation dyeing, using an oxidizing system other than hydrogen peroxide.

The inventor has unexpectedly discovered that it is possible to achieve this aim by using at least one oxidation dye precursor, at least one alcohol oxidase enzyme, at least one substrate for the said enzyme, and at least one oxyalkylenated anionic surfactant, in compositions for dyeing keratin fibers, for example human keratin fibers, such as the hair.

The compositions according to the present disclosure allow the production of highly homogeneous formulations which, once applied, respect the nature of the keratin fibers and do not have the solubilization and crystallization problems encountered for example with the uric acid/uricase system. The inventor has also noted that the stability of the compositions according to the present disclosure, for example the stability of the alcohol oxidase enzyme of these compositions, is improved.

Use of compositions according to the present disclosure to dye keratin fibers can lead to the production of strong, unselective, and fast colors. These compositions are capable of generating varied shades of intense and uniform color, without any significant degradation of the keratin fibers. In addition, the use of such compositions can improve the hold of permanent-waved hair and reduce the porosity of the hair.

Other characteristics, aspects, subjects, and advantages of the present disclosure will become evident upon reading the description and the examples that follow.

Examples of oxyalkylenated anionic surfactants that may be used, alone or as mixtures, in the context of the present disclosure, include, but are not limited to, polyoxyalkylenated carboxylic acid ethers and salts thereof, polyoxyalkylenated (C₆-C₂₄)alkyl(C₆-C₂₄)arylether-carboxylic acids, polyoxyalkylenated (C₆-C₂₄)alkylamidoether-carboxylic acids and salts thereof, for example those comprising from 2 to 50 ethylene oxide groups, and mixtures thereof. The anionic surfactants of the acid type or polyoxyalkylenated carboxylic ether salt type may be those corresponding to formula (I) below:
R₁—(OC₂H₄)ₙ—OCH₂COOA (1)
wherein:
R₁ is chosen from alkyl and alkylaryl groups, wherein the alkyl groups comprise from 6 to 20 carbon atoms, and aryl may be chosen from phenyl;
n is chosen from integers and decimal numbers, and has a mean value ranging for example from 2 to 24, such as from 3 to 10; and
A is chosen from hydrogen, ammonium, sodium, potassium, lithium, magnesium, and monoethanolamine and triethanolamine residues.

In one embodiment, mixtures of the oxyalkylenated anionic surfactants described above may be used. Mixtures of compounds of formula (1), for example mixtures in which the groups R₁ are different, may also be used.

In the context of the present disclosure, the at least one alcohol oxidase enzyme that may be used in the dye compositions belongs to the class E.C.1.1.3 of the enzyme nomenclature (see Enzyme Nomenclature, Academic Press, Inc., 1992).

The at least one enzyme may be chosen from primary alcohol oxidases (EC1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38), and aromatic alcohol oxidases (EC 1.1.3.7), also known as aryl alcohol oxidases.

According to one embodiment, the at least one enzyme used in the compositions according to the present disclosure may be a primary alcohol oxidase (EC1.1.3.13).

Alcohol oxidase enzymes form a class of 2-electron oxidoreductase enzymes.

The at least one alcohol oxidase enzyme used in the dye compositions according to the present disclosure may be derived from an extract of plants, of animals, of microorganisms, for example bacteria, fungi, yeast, microalgae or viruses, of differentiated or undifferentiated cells, obtained in vivo or in vitro, unmodified or genetically modified, or synthetic (obtained by chemical or biotechnological synthesis).

By way of non-limiting example, the at least one alcohol oxidase enzyme may be derived from one of the following species: Rhodococcus erythropolis and Pseudomonas pseudoalcaligenes, which are bacteria; Aspergillus niger, Kamagataella pastoris, Phanerochaete chrysosporium, Polyporus obtusus, Hansenula polymorpha, Poria contigua, Penicillium simplicissimum, and Pleurotus pulmonarius, which are fungi; Pichia sp. (pastoris, methanolica, angusta) and Candida sp. (boidinii, albicans, tropicalis), which are yeasts; Pinus strobus, which is a plant; and Gastropode mollusc and Manduca sexta, which are animals.

In one embodiment, the at least one enzyme used in the compositions according to the present disclosure is an alcohol oxidase derived from Pichia pastoris.

The at least one alcohol oxidase enzyme may be present in the dye compositions in an amount ranging from 0.05% to 20% by weight, relative to the total weight of the composition, for example, from 0.1% to 10% by weight, relative to the weight of the composition, such as from 0.5% to 8% by weight, relative to the weight of the composition.

The enzymatic activity of the at least one alcohol oxidase enzyme used in accordance with the present disclosure may be defined from the oxidation of the donor under aerobic conditions. One unit U is the amount of enzyme leading to the generation of 1 μmol of hydrogen peroxide per minute at a given pH and at a temperature of 25° C.

The at least one alcohol oxidase may be present in the compositions according to the present disclosure in an amount ranging from 10³ U to 10⁵ U, for example from 2×10³ U to 5×10⁴ U, per 100 g of dye composition.

The at least one substrate for the enzyme is also known as a donor for the enzyme. The at least one substrate for the enzyme in the compositions of the present disclosure may be an alcohol chosen from primary alcohols, secondary alcohols, long-hydrocarbon-chain alcohols, and aromatic alcohols. Non-limiting examples of the at least one donor for the primary alcohol oxidases include primary alcohols comprising from 1 to 6 carbon atoms. Non-limiting examples of the at least one donor for the aryl alcohol oxidases include benzyl alcohol, 4-tert-butylbenzyl alcohol, 3-hydroxy-4-methoxybenzyl alcohol, veratryl alcohol, 4-methoxybenzyl alcohol, and cinnamyl alcohol. 2,4-hexadien-1-ol may also be used as the at least one donor for the aryl alcohol oxidases.

According to another aspect of the present disclosure, the at least one substrate for the enzyme may be a compound bearing at least one aliphatic or aromatic alcohol functional group, which is suitable for reacting with the enzyme used. The compound bearing at least one aliphatic or aromatic alcohol functional group may be, for example, an oxidation dye precursor or a cosmetically acceptable adjuvant, such as a polymer, a surfactant or a preserving agent bearing at least one alcohol functional group. As a further example, the at least one substrate for the enzyme may be an oxidation dye precursor bearing at least one aliphatic or aromatic alcohol functional group. For example, N-(β-hydroxypropyl)-para-phenylenediamine, which bears a primary alcohol functional group, may serve as an oxidation base and as the at least one substrate for the alcohol oxidase. Similarly, oxidation couplers, such as meta- or para-aminophenol, may fulfil both functions. Such precursors are described hereinbelow. In this aspect of the present disclosure, the use of other substrates for the enzyme is optional.

Thus, the present disclosure relates to compositions for dyeing keratin fibers, for example human keratin fibers such as the hair, comprising, in a medium that is suitable for dyeing, at least one oxidation dye precursor; at least one alcohol oxidase enzyme; at least one substrate, bearing an alcohol functional group, for the enzyme; and at least one oxyalkylenated anionic surfactant, the at least one substrate optionally being substituted (i.e. replaced) totally or partially with the at least one oxidation dye precursor in the case where the at least one oxidation dye precursor bears at least one functional group chosen from aliphatic and aromatic alcohol functional groups.

Use of the compositions in accordance with the present disclosure may reduce the risks associated with handling hydrogen peroxide. Furthermore, the concentration of preserving agents in the compositions according to the present disclosure may be reduced by using compounds comprising at least one alcohol functional group that also have preserving properties.

The at least one substrate for the enzyme may be present in an amount ranging from 0.01% to 60% by weight, relative to the total weight of the composition, for example from 0.05% to 30% by weight, relative to the total weight of the composition.

The at least one oxidation dye precursor used in the compositions according to the present disclosure may be any art-recognized oxidation base. Oxidation bases may be chosen, for example, from para-phenylenediamines, bis(phenyl)alkylenediamines, para-aminophenols, ortho-aminophenols and heterocyclic bases, and the addition salts thereof.

Among the para-phenylenediamines, non-limiting mention may be made, by way of example, of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl 2-methylaniline, 4-amino-2-chloro-N,N-bis(β-hydroxyethyl)aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl) pyrrolidine, and the acid addition salts thereof.

Among the para-phenylenediamines mentioned above, further non-limiting examples include para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the acid addition salts.

Among the bis(phenyl)alkylenediamines, non-limiting mention may be made, by way of example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the acid addition salts.

Among the para-aminophenols, non-limiting mention may be made, by way of example, of para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, 4-amino-2,6-dichlorophenol, 4-amino-6[(5′-amino-2′-hydroxy-3′-methyl)phenyl methyl]-2-methylphenol and bis(5′-amino-2′-hydroxyl)phenylmethane, and the acid addition salts thereof.

Among the ortho-aminophenols, non-limiting mention may be made, by way of example, of 2-aminophenol, 2-amino-5-methylphenol, 2-amino-6-methylphenol and 5-acetamido-2-aminophenol, and the acid addition salts thereof.

Among the heterocyclic bases, non-limiting mention may be made, by way of example, of pyridine derivatives, pyrimidine derivatives, and pyrazole derivatives.

Among the pyridine derivatives, non-limiting mention may be made of the compounds described, for example, in British Patent Nos. GB 1,026,978 and GB 1,153,196, such as 2,5-diaminopyridine, 2-(4-methoxyphenyl)amino-3-aminopyridine, 3,4-diaminopyridine, and the acid addition salts thereof.

Other pyridine oxidation bases that may be used in the present disclosure include, but are not limited to, the 3-aminopyrazolo[1,5-a]pyridine oxidation bases and the addition salts thereof described, for example, in French Patent Application No. FR 2,801,308. By way of example, non-limiting mention may be made of pyrazolo[1,5-a]pyrid-3-ylamine; 2-acetylaminopyrazolo[1,5-a]pyrid-3-ylamine; 2-morpholin-4-ylpyrazolo[1,5-a]pyrid-3-ylamine; 3-aminopyrazolo[1,5-a]pyrid ine-2-carboxylic acid; 2-methoxypyrazolo[1,5-a]pyrid-3-ylamine; (3-aminopyrazolo[1,5-a]pyrid-7-yl)methanol; 2-(3-aminopyrazolo[1,5-a]pyrid-5-yl)ethanol; 2-(3-aminopyrazolo[1,5-a]pyrid-7-yl)ethanol; (3-aminopyrazolo[1,5-a]pyrid-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyrid-3-ylamine; pyrazolo[1,5-a]pyridine-3,5-diamine; 5-morpholin-4-ylpyrazolo[1,5-a]pyrid-3-ylamine; 2-[(3-aminopyrazolo[1,5-a]pyrid-5-yl)(2-hydroxyethyl)amino]ethanol; 2-[(3-aminopyrazolo[1,5-a]pyrid-7-yl)(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyrid-6-ol; 3-aminopyrazolo[1,5-a]pyrid-7-ol; and the acid addition salts thereof.

Among the pyrimidine derivatives, non-limiting mention may be made of the compounds described, for example, in German Patent No. DE 2,359,399; Japanese Patent Nos. JP 88-169,571 and JP 05,163,124; European Patent No. EP 0,770,375 or International Patent Publication No. WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine, and 2,5,6-triaminopyrimidine, the addition salts thereof, and the tautomeric forms thereof when a tautomeric equilibrium exists.

Among the pyrazole derivatives, non-limiting mention may be made of the compounds described in German Patent Nos. DE 3,843,892 and DE 4,133,957, International Patent Publication Nos. WO 94/08969 and WO 94/08970, French Patent Application No. FR-A-2,733,749, and German Patent Application No. DE 19,543,988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropyl pyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and the addition salts thereof.

The oxidation base may be present in an amount ranging from 0.0001% to 20%, by weight, for example from 0.005% to 6%, by weight, relative to the total weight of the composition.

The at least one oxidation dye precursor used in the compositions according to the present disclosure may be any art-recognized oxidation coupler. Among oxidation couplers, non-limiting mention may be made of meta-phenylenediamines, meta-aminophenols, meta-diphenols, naphthalenic couplers and heterocyclic couplers, and the addition salts thereof.

Non-limiting examples that may be mentioned include 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, and the addition salts thereof.

The oxidation coupler may be present in an amount ranging from 0.0001% to 20%, by weight, for example from 0.005% to 6%, by weight, relative to the total weight of the composition.

The acid addition salts that may be used for the oxidation bases and couplers are chosen, for example, from the hydrochlorides, hydrobromides, sulfates, citrates, succinates, tartrates, lactates, tosylates, benzenesulfonates, phosphates, and acetates.

The addition salts that may be used in the context of the present disclosure may be chosen, for example, from the addition salts with sodium hydroxide, potassium hydroxide, ammonia, amines, and alkanolamines.

The dye compositions in accordance with the present disclosure may further comprise at least one direct dye that may be chosen, for example, from neutral, acidic, and cationic nitrobenzene dyes; neutral, acidic, and cationic azo direct dyes; neutral, acidic, and cationic quinone, such as anthraquinone, direct dyes; azine direct dyes; methine, azomethine, triarylmethane, and indoamine direct dyes; and natural direct dyes. The at least one additional direct dye may be chosen, for instance, from cationic direct dyes and natural direct dyes.

Among the cationic direct dyes that may be used according to the present disclosure, non-limiting mention may be made of the cationic azo direct dyes described in International Patent Publication Nos. WO 95/15144 and WO 95/01772, and European Patent Application No. EP 714,954.

Among these compounds, non-limiting mention may be made, for example, of the following dyes:
1,3-dimethyl-2-[[4-(dimethylamino)phenyl]azo]-1H-imidazolium chloride,
1,3-dimethyl-2-[(4-aminophenyl)azo]-1H-imidazolium chloride, and
1-methyl-4-[(methyl phenyl hydrazino)methyl]pyrid inium methyl sulfate.

Among the natural direct dyes that may be used according to the present disclosure, non-limiting mention may be made of lawsone, juglone, alizarin, purpurin, carminic acid, kermesic acid, purpurogallin, protocatechaldehyde, indigo, isatin, curcumin, spinulosin, and apigenidin. Extracts or decoctions comprising these natural dyes, for instance henna-based poultices and extracts, may also be used.

The at least one direct dye may be present in an amount ranging from 0.001% to 20% by weight, for example from 0.005% to 10% by weight, relative to the total weight of the ready-to-use composition.

The dye compositions in accordance with the present disclosure may further comprise at least one adjuvant conventionally used in compositions for dyeing keratin fibers, such as antioxidants, penetrating agents, sequestering agents, fragrances, buffers, dispersants, surfactants, conditioners such as, for example, volatile and non-volatile, modified and unmodified silicones, cationic polymers, cations, film-forming agents, ceramides, preserving agents, opacifiers, vitamins, provitamins, and thickening polymers.

Each adjuvant may be present in an amount ranging from 0.01% to 20% by weight relative, to the weight of the composition.

Needless to say, the person skilled in the art will take care to select this or these optional additional compound(s) such that the beneficial properties intrinsically associated with the oxidation dye compositions in accordance with the present disclosure are not, or are not substantially, adversely affected by the envisaged addition(s).

The medium that is suitable for dyeing, also known as the dye support, may consist of water or may comprise a mixture of water and at least one organic solvent to dissolve the compounds that would not be sufficiently water-soluble. As appropriate, this at least one organic solvent may be a substrate of the at least one alcohol oxidase enzyme, such as ethanol or isopropanol. It may also be a compound other than a substrate of the at least one alcohol oxidase enzyme, chosen from, for example, polyol ethers, for instance 2-butoxyethanol, propylene glycol, propylene glycol monomethyl ether, diethylene glycol monomethyl ether, diethylene glycol monoethyl ether, and phenoxyethanol.

The at least one organic solvent may be present, for example, in an amount ranging from 1% to 40% by weight, relative to the total weight of the dye composition, such as from 5% to 30% by weight, relative to the total weight of the dye composition.

The pH of the dye compositions in accordance with the present disclosure may range from 6 to 11, for example from 7 to 10. It may be adjusted for the desired value using at least one acidifying or basifying agent usually used in the dyeing of keratin fibers, or alternatively using standard buffer systems.

Among the acidifying agents that may be used, non-limiting mention may be made, for example, of mineral and organic acids, for instance hydrochloric acid, orthophosphoric acid, sulfuric acid, carboxylic acids, such as acetic acid, tartaric acid, citric acid and lactic acid, and sulfonic acids.

Among the basifying agents that may be used, non-limiting mention may be made, for example, of aqueous ammonia, alkyl metal carbonates, alkanolamines such as monoethanolamine, diethanolamine, triethanolamine, and derivatives thereof, sodium hydroxide, potassium hydroxide, and the compounds of formula (2) below: 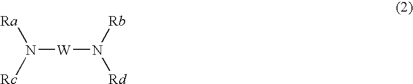
wherein:
W is chosen from propylene residues optionally substituted with a hydroxyl group or a C₁-C₄ alkyl radical; and
Ra, Rb, Rc, and Rd, which may be identical or different, are chosen from hydrogen and C₁-C₄ alkyl and C₁-C₄ hydroxyalkyl radicals.

The dye compositions may be in various forms, such as in the form of thickened liquids, creams or gels, or in any other form that is suitable for dyeing keratin fibers, such as human hair.

When the at least one oxidation dye and the at least one alcohol oxidase are present in the same ready-to-use composition, the composition is, for example, free of oxygen gas, so as to avoid any premature oxidation of the at least one oxidation dye.

The present disclosure also relates to processes for dyeing keratin fibers, for example human keratin fibers, such as the hair, in which at least one dye composition according to the present disclosure is applied to these fibers, the minimum duration of this application being a period of time that is sufficient to develop the desired coloration.

The color is then revealed by bringing into contact the alcohol oxidase enzyme and its substrate, in the presence of oxygen.

In one embodiment, a composition according to the present disclosure is applied to keratin fibers. After leaving it to act for a period of time ranging from 3 to 60 minutes, for example from 5 to 40 minutes, the keratin fibers are rinsed, washed with shampoo, rinsed again and then dried.

In another aspect of the present disclosure, the dye compositions may be ready-to-use compositions comprising, in a medium that is suitable for dyeing keratin fibers, at least one oxidation dye precursor, at least one alcohol oxidase enzyme, at least one substrate for the said enzyme, and at least one oxyalkylenated anionic surfactant, and the composition may be stored in anaerobic form, free of oxygen gas.

According to one embodiment, the processes for dyeing keratin fibers comprise separately storing a composition (A) comprising, in a medium that is suitable for dyeing keratin fibers, at least one oxidation dye precursor and a composition (B) comprising, in a medium that is suitable for dyeing keratin fibers, at least one alcohol oxidase enzyme, wherein composition (A) and/or composition (B) comprise at least one substrate for the said enzyme, and composition (A) and/or composition (B) comprise at least one oxyalkylenated anionic surfactant, and mixing together compositions (A) and (B) at the time of use before applying this mixture to keratin fibers.

According to another embodiment of the present disclosure, the processes for dyeing keratin fibers comprise separately storing a composition (A) comprising, in a medium that is suitable for dyeing keratin fibers, at least one oxidation dye precursor, at least one substrate for the said enzyme, and at least one oxyalkylenated anionic surfactant and a composition (B) comprising, in a medium that is suitable for dyeing keratin fibers, at least one alcohol oxidase enzyme, and mixing together compositions (A) and (B) at the time of use before applying this mixture to keratin fibers.

The color may be revealed at acidic, neutral, or alkaline pH. In the case where the process is performed using a composition (A) comprising at least one oxidation dye precursor, at least one substrate for the said enzyme, and at least one oxyalkenylated anionic surfactant and a composition (B) comprising at least one alcohol oxidase enzyme, the enzyme may be added to the composition of the present disclosure just at the time of use, or it may be used starting with a composition comprising it, applied simultaneously or sequentially to the composition of the present disclosure.

Composition (B), also called oxidizing composition, may also comprise at least one adjuvant conventionally used in compositions for dyeing keratin fibers, such as hair, and as defined above.

The pH of composition (B), also called oxidizing composition, is such that, after mixing with dye composition (A), the pH of the resultant composition applied to the keratin fibers may range, for example, from 6 to 11, such as from 7 to 10. It may be adjusted to the desired value by means of acidifying or basifying agents usually used in the dyeing of keratin fibers and as defined above.

The application of the compositions according to the present disclosure may be carried out at a temperature ranging from room temperature to 220° C., for example from room temperature to 60° C.

The present disclosure also relates to multi-compartment devices or dyeing “kits”, wherein a first compartment comprises composition (A) as defined above and a second compartment comprises composition (B) as defined above. These kits may be equipped with a means for applying the desired mixture to keratin fibers, such as the devices described in French Patent No. FR-2,586,913.

The present disclosure may be understood more clearly with the aid of the non-limiting examples that follow, which constitute preferred embodiments of the compositions according to the disclosure. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLES

The following composition was prepared in accordance with the present disclosure:

| Constituent | Composition 1 | |
|---|---|---|
| AKYPO RLM 45 CA | 7% | A.M. |
| (Kao - 90% AM) | | |
| Ethanol | 25 | g |
| para-Phenylenediamine | 3 × 10⁻³ | mol |
| meta-Aminophenol | 3 × 10⁻³ | mol |
| Alcohol oxidase | 20 000 | units |
| 2-Amino-2-methyl-1-propanol | qs pH 7 | |
| Distilled water | qs 100 | g |

The alcohol oxidase used is the enzyme sold by the company Biozyme Laboratories, in liquid form at a concentration of 1980 units/ml.

The unit U corresponds to the amount of enzyme leading to the generation of 1 μmol of hydrogen peroxide per minute at pH 7.5 (100 mM phosphate buffer) and at a temperature of 25° C.

The above compositions were applied to locks of natural grey permanent-waved hair containing 90% white hairs, and left to act for 30 minutes. The bath ratio was set at 5. The alcohol oxidase was added extemporaneously. The hair was then rinsed, washed with a standard shampoo and then dried.

A fast and homogeneous coloration in khaki-green shades was obtained.

1. A composition for dyeing keratin fibers comprising, in a medium that is suitable for dyeing, at least one oxidation dye precursor, at least one alcohol oxidase enzyme, at least one substrate for the enzyme, and at least one oxyalkylenated anionic surfactant,
wherein the at least one substrate is optionally totally or partially substituted with the at least one oxidation dye precursor if the at least one oxidation dye precursor bears at least one functional group chosen from aromatic and aliphatic alcohol functional groups.

2. The composition according to claim 1, wherein the at least one oxyalkylenated anionic surfactant is chosen from polyoxyalkylenated carboxylic acid ethers and salts thereof, polyoxyalkylenated (C₆-C₂₄)alkyl(C₆-C₂₄)arylether-carboxylic acids, polyoxyalkylenated (C₆-C₂₄)alkylamidoether-carboxylic acids and salts thereof, and mixtures thereof.

3. The composition according to claim 2, wherein the at least one oxyalkylenated anionic surfactant is chosen from compounds of formula (1):
R₁—(OC₂H₄)ₙ—CH₂COOA (1)
wherein:
R₁ is chosen from alkyl and alkylaryl groups, wherein the alkyl radical comprises from 6 to 20 carbon atoms;
n is chosen from integers and decimal numbers ranging from 2 to 24; and
A is chosen from hydrogen, ammonium, sodium, potassium, lithium, magnesium, and monoethanolamine and triethanolamine residues.

4. The composition according to claim 1, wherein the at least one alcohol oxidase enzyme is chosen from primary alcohol oxidases (EC1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38), and aromatic alcohol oxidases (EC 1.1.3.7).

5. The composition according to claim 4, wherein the at least one alcohol oxidase enzyme is derived from one of the following species: Rhodococcus erythropolis, Pseudomonas pseudoalcaligenes, Aspergillus niger, Kamagataella pastoris, Phanerochaete chrysosporium, Polyporus obtusus, Hansenula polymorpha, Poria contigua, Penicillium simplicissimum, Pleurotus pulmonarius, Pichia sp., Candida sp., Pinus strobus, Gastropode mollusc, and Manduca sexta.

6. The composition according to claim 1, wherein the at least one alcohol oxidase enzyme is present in an amount ranging from 0.05% to 20% by weight, relative to the total weight of the composition.

7. The composition according to claim 1, wherein the at least one substrate for the enzyme is an alcohol chosen from primary alcohols, secondary alcohols, long-hydrocarbon-chain alcohols, and aromatic alcohols.

8. The composition according to claim 7, wherein the alcohol is present in an amount ranging from 0.01% to 60% by weight, relative to the total weight of the composition.

9. The composition according to claim 1, wherein the at least one oxidation dye precursor is an oxidation base chosen from para-phenylenediamines, bis(phenyl)alkylene-diamines, para-aminophenols, ortho-aminophenols, heterocyclic bases, and the addition salts thereof.

10. The composition according to claim 9, wherein the oxidation base is present in an amount ranging from 0.0001% to 20% by weight, relative to the total weight of the composition.

11. The composition according to claim 1, wherein the at least one oxidation dye precursor is an oxidation coupler chosen from meta-phenylenediamines, meta-aminophenols, meta-diphenols, naphthalenic couplers, heterocyclic couplers, and the addition salts thereof.

12. The composition according to claim 11, wherein the oxidation coupler is present in an amount ranging from 0.0001% to 20% by weight, relative to the total weight of the composition.

13. The composition according to claim 1, further comprising at least one direct dye chosen from cationic and natural direct dyes.

14. A process for dyeing keratin fibers, comprising
applying to the fibers at least one dye composition comprising, in a medium that is suitable for dyeing, at least one oxidation dye precursor, at least one alcohol oxidase enzyme, at least one substrate for the enzyme, and at least one oxyalkylenated anionic surfactant,
wherein the at least one substrate is optionally totally or partially substituted with the at least one oxidation dye precursor if the at least one oxidation dye precursor bears at least one functional group chosen from aromatic and aliphatic alcohol functional groups; and
leaving the composition on the fibers for a period of time that is sufficient to develop the desired coloration.

15. A process for dyeing keratin fibers, comprising
applying to the fibers a ready-to-use composition comprising, in a medium that is suitable for dyeing keratin fibers, at least one dye composition comprising at least one oxidation dye precursor, at least one alcohol oxidase enzyme, at least one substrate for the enzyme, and at least one oxyalkylenated anionic surfactant,
wherein the at least one substrate is optionally totally or partially substituted with the at least one oxidation dye precursor if the at least one oxidation dye precursor bears at least one functional group chosen from aromatic and aliphatic alcohol functional groups, wherein the ready-to-use composition is stored in anaerobic form, free of oxygen gas; and
leaving the composition on the fibers for a period of time that is sufficient to develop the desired coloration.

16. The process according to claim 15, further comprising a preliminary step comprising
separately storing a composition (A) comprising, in a medium that is suitable for dyeing keratin fibers, at least one oxidation dye precursor and a composition (B) comprising, in a medium that is suitable for dyeing keratin fibers, at least one alcohol oxidase enzyme, wherein the composition (A) and/or the composition (B) comprise at least one substrate for the at least one alcohol oxidase enzyme, and the composition (A) and/or the composition (B) comprise at least one oxyalkylenated anionic surfactant; and
mixing together compositions (A) and (B) at the time of use before applying this mixture to the keratin fibers.

17. The process according to claim 16, wherein the preliminary step comprises
separately storing a composition (A) comprising, in a medium that is suitable for dyeing keratin fibers, at least one oxidation dye precursor, at least one substrate for the at least one alcohol oxidase enzyme, and at least one oxyalkylenated anionic surfactant and a composition (B) comprising, in a medium that is suitable for dyeing keratin fibers, at least one alcohol oxidase enzyme; and
mixing together compositions (A) and (B) at the time of use before applying this mixture to the keratin fibers.

18. A multi-compartment dyeing kit, comprising at least one first compartment comprising a composition (A) comprising, in a medium that is suitable for dyeing keratin fibers, at least one oxidation dye precursor and at least one second compartment comprising a composition (B) comprising, in a medium that is suitable for dyeing keratin fibers, at least one alcohol oxidase enzyme,
wherein the composition (A) and/or the composition (B) comprise at least one substrate for the at least one alcohol oxidase enzyme, and the composition (A) and/or the composition (B) comprise at least one oxyalkylenated anionic surfactant.

19. The multi-compartment dyeing kit according to claim 18, wherein the at least one first compartment comprises a composition (A) comprising, in a medium that is suitable for dyeing keratin fibers, at least one oxidation dye precursor, at least one substrate for the at least one alcohol oxidase enzyme, and at least one oxyalkylenated anionic surfactant and the at least one second compartment comprises a composition (B) comprising, in a medium that is suitable for dyeing keratin fibers, at least one alcohol oxidase enzyme.
